(19) United States
(12) Patent Application Publication
(10) Pub. No.: US 2009/0202467 A1
(43) Pub. Date: Aug. 13, 2009

(54) SCLEROTHERAPY FOR VARICOSE VEINS

(76) Inventor: Richard W. Bock, Asheville, NC (US)

Correspondence Address: JAMES REMENICK;NOVAK DRUCE & QUIGG, LLP, 1300 I STREET NW, SUITE 1000 WEST TOWER, WASHINGTON, DC, 20005, US

(21) Appl. No.: 12/335,084

(22) Filed: Dec. 15, 2008

Related U.S. Application Data
(60) Provisional application No. 61/027,226, filed on Feb. 8, 2008.

(51) Int. Cl. A61K 31/77; C08G 65/04; A61P 9/00

(52) U.S. Cl. 424/78.38; 525/93; 521/149

(57) ABSTRACT

This invention is directed to pharmaceutical compositions and methods of treating varicose veins, including telangiectasias and reticular veins, and related symptoms and diseases. Embodiments comprise injection into a varicose vein of an amphiphilic block copolymer which has properties of conversion from liquid to gel state, surfactant properties, or combinations thereof. Other embodiments consist of the combination of an amphiphilic block copolymer with a co-solvent, also allowing the injected combination to form a gel within the vein. In other embodiments, an amphiphilic block copolymer is combined with a co-solvent, a sclerosant, a vasoconstrictor, water, or combinations thereof. Other embodiments of the invention include a combination of a sclerosant such as sodium tetradecyl sulfate or polidocanol with a vasoconstrictor, preferably a long-acting vasoconstrictor such as oxymetazoline. Other embodiments are directed to compositions and methods of treating venous and arteriovenous malformations and cancer.

### REFERENCE TO RELATED APPLICATION

This invention claims priority to U.S. Provisional Application No. 61/027,226 entitled “Sclerotherapy for Varicose Veins” filed Feb. 8, 2008, the entirety of which is hereby incorporated by reference.

### BACKGROUND

1. Field of Invention

This invention is directed to compositions and methods of treating varicose veins and related symptoms and diseases.

2. Description of the Background

Normal veins carry deoxygenated blood from the periphery to the heart. In the legs, valves reside in many veins to assist in directing flow upward out of the legs and toward the heart. Varicose veins are enlarged or twisted veins which develop when the valves malfunction, allowing reverse flow (reflux). Blood collects and pools in the lower veins, most commonly in the superficial leg veins, causing the veins to become enlarged and damaged.

Varicose veins affect women in a higher percentage than men. Varicose Vein Disease (VVD) has been linked to heredity, but some factors may speed its development, including pregnancy, obesity, menopause, aging, prolonged standing, physical trauma, and hormone levels.

Varicose Vein Disease describes a broad spectrum of disorders and symptoms, such as spider veins, telangiectasias, reticular varicose veins, bulging varicose vein tributaries, and varicose saphenous veins. Besides an undesirable visual appearance, symptoms of varicose veins include pain, heaviness, fatigue, burning, itching, swelling, throbbing, and tenderness in the surrounding areas of the body. Edema (swelling caused by fluid accumulation) can occur, and the skin may become discolored, exhibiting a brownish-blue shiny appearance around the veins. The skin over the veins may also turn dry and itchy, and it frequently darkens (stasis dermatitis) because of the waste products and fluid pressure building up in the legs. Loss of skin and open ulcers sometimes follow.

Spider veins are not only cosmetically bothersome, but they can cause burning, stinging and itching. Reticular varicosities also cause cosmetic problems, in addition to localized aching. Bulging varicose vein tributaries cause localized aching and generalized fatigue. Diseased saphenous veins cause deep, aching calf pain, and edema, which can lead to skin changes and ulcer formation as well as debility.

Minor injuries to the areas around varicose veins may bleed more than normal or take a longer time to heal than they would normally. More serious symptoms can also develop if left untreated, including inflammation (phlebitis), blood clots, ankle sores or skin ulcers and bleeding. Varicose veins usually enlarge and worsen over time. Legs and feet may begin to swell and sensations of pain, heaviness, burning or tenderness may occur. Ultimately, VVD-related symptoms can become chronic, debilitating and costly.

The general term “varicose vein” encompasses a range of diseased veins. Telangiectasias (also called spider veins, or spider telangiectasias) generally range from 0.3 mm to 1 mm, while reticular veins and venulectasias typically measure between 1 mm to 4 mm. Larger varicose veins include tributary varicosities as well as varicose greater and lesser saphenous veins. Telangiectasias (spider veins) are the most common type of varicose veins. They are seen in up to 50% of women over 40 years of age.

Since most of the blood in the legs is returned by the deep veins, and the superficial veins only return about 10%, superficial veins can be removed or ablated without serious harm. The traditional treatment for larger varicose veins has been vein stripping to remove affected saphenous veins or phlebectomy for removal of bulging tributary varicose veins.

Non-surgical treatments for larger varicose veins include phlebectomy, microphlebectomy, endovenous laser ablation, and radiofrequency vein ablation. Sclerotherapy using liquid or foamed sclerosing agents is a common treatment for smaller varicose veins. However, patient satisfaction after sclerotherapy can be as low as only 40 to 60%.

### Sclerotherapy

Despite its current shortcomings, sclerotherapy is a rapidly growing field. A sclerosing agent is typically injected into the vein with small needle in an outpatient setting, generally in a dedicated vein treatment clinic or office. One agent used in sclerotherapy can be hypertonic saline. Approved as an abortifacent, it kills intimal cells (the cells lining the interior of all veins and arteries) via dehydration. Although its use is still favored by some dermatologists, the procedure is painful, often ineffective, and can cause ulceration of the skin.

Another sclerotherapy agent is 2-[2-[2-[2-[2-[2-[2-[2-[2-(Dodecyloxy)ethoxy]ethoxy]ethoxy]ethoxy]ethoxy]ethoxy]ethoxy]ethoxy]ethanol (hereinafter, “polidocanol”), which is a surfactant or detergent. Polidocanol can cause skin staining and ulceration, produces only variable effectiveness, and most authorities recommend that compression stockings be worn for days or weeks after each treatment session.

Sodium tetradecyl sulfate (STS), available in the U.S. as SOTRADECOL®, a trademark of Cumberland Pharmaceuticals, Inc., is the most viable sclerotherapy agent currently available. STS, is a surfactant or a detergent. SOTRADECOL® is commercially available from AngioDynamics, Inc. Treatment with compression stockings is also recommended after treatment with STS.

Common complications of sclerotherapy include failure to sclerose (e.g. when the concentration is too dilute), bruising, inconvenience and aggravation (for example as related to the hot, unwieldy and unattractive thigh- or waist-high compression stockings recommended for 1 to 3 weeks after treatment), trapped blood (requiring follow-up visits and painful drainage of the clot), telangiectatic matting, and skin staining.

Telangiectatic matting occurs after otherwise successful sclerotherapy. The treated area becomes covered with a fine network of red blood vessels which are apparent to the naked eye. These vessels are smaller than spider telangiectasias and are very difficult to treat. Telangiectatic matting has also been linked to thrombus formation associated with sclerotherapy

The most common long-lasting complication of current sclerotherapy procedures is skin staining, which typically comprises brownish blotches in the areas where the sclerotherapy was successful in producing obliteration of the veins. Skin staining affects up to 70% of sclerotherapy patients, and although it often fades, it may take up to a year to disappear. One mechanism for skin staining comprises the following: a surfactant or detergent destroys intima of the vein; the integrity of vein wall is compromised and/or destroyed; blood leaks out into tissue and persists as clotted blood (thrombus); hemoglobin is degraded to hemosiderin (brown); hemosiderin is insoluble and becomes trapped; macrophages phagocytose hemosiderin; macrophages remain in skin over years, only slowly migrating away. Another mechanism for skin staining includes the following: a surfactant/detergent destroys the intima of the vein, but then blood refills the vein within minutes. Thrombus (clotted blood) forms inside the sclerosant-damaged vein; hemoglobin in the blood is degraded to hemosiderin (brown); hemosiderin is insoluble, becomes trapped; macrophages phagocytose hemosiderin; macrophages remain in skin over years, only slowly migrating away. The critical event common to both pathways is the formation of unwanted thrombus either inside or adjacent to the treated varicose vein.

Thus, although sclerotherapy can be an effective and relatively noninvasive treatment for varicose veins and VVD generally, the two most common complications of this treatment are actual failure to sclerose and hyperpigmentation. Ineffectiveness can be the result of using too low of a concentration of sclerosant, and skin staining is often the result of using too high of a concentration. The therapeutic margin is thus very narrow, and effective concentrations can vary from patient to patient and even from vein to vein within the same patient. Veins may fail to sclerose because the concentration of sclerosant is too small or when too high of a sclerosant concentration, the skin will show hyperpigmentation or stained skin. This narrow therapeutic margin problem is further exacerbated by the fact that the concentration of a liquid sclerosant drops as a function of the distance from the point of injection. In other words, in the vein right at the injection site, the effective concentration is very close to the concentration of the drug in the syringe. But as the drug flows in, it mixes with blood, and the concentration falls even millimeters away from the injection site. Drug dilution also occurs over time, as blood flows back (even at the injection point) and dilutes the liquid drug.

### Poloxamers

Poloxamers, also known as PLURONIC® or PLURONICS®, trademarks of BASF SE, are amphiphilic nonionic block copolymers. These polymers are commonly named with the word “poloxamer” followed by a number to indicate which polymer is being discussed (e.g. Poloxamer 407). Throughout this application, the term “poloxamer” may be used interchangeably with “poloxamine” or PLURONIC®, or amphiphilic block copolymer to refer to this class of substances.

Poloxamers are composed of a central hydrophobic chain of polyoxypropylene (poly-propylene oxide) flanked by two hydrophilic chains of polyoxyethylene (polyethylene oxide). National Formulary grades exist for various poloxamers.

Poloxamer 124, as described in the National Formulary, is also known as PLURONIC L44®.

Poloxamer 188, as described in the National Formulary, is also known as PLURONIC F68®.

Poloxamer 237, as described in the National Formulary, is also known as PLURONIC F87®.

Poloxamer 338, as described in the National Formulary, is also known as PLURONIC F108®.

Poloxamer 407, as described in the National Formulary, is also known as PLURONIC F127®.

Poloxamers have been used in medicine to treat burns (Poloxamer-407, as described in the National Formulary), sickle cell anemia (Poloxamer 188, as described in the National Formulary), for splenotrophic particles (Poloxamine-908, as described in the National Formulary), in chemotherapy drug delivery and in rectal, nasal or subcutaneous drug delivery (multiple poloxamers), in therapeutic temporary embolization of renal and mesenteric arteries (PLURONIC F127®), as a vehicle for the subcutaneous or intramuscular delivery of vaccines and other drugs (PLURONIC F127® and others), in off-pump coronary bypass procedures (PLURONIC F127®), and in many preparations for delivery of drugs which would be insoluble without being combined with a poloxamer, or in order to provide slow release of drugs injected into the subcutaneous fat or muscle, admixed with a poloxamer that gels upon reaching body temperature. However, poloxamers are not believed to have been heretofore used as pharmaceuticals or especially for sclerotherapy.

### SUMMARY OF THE INVENTION

The present invention overcomes the problems and disadvantages associated with existing treatments for varicose vein disease, and provides a new class of highly efficient pharmaceutical compositions, agents, treatments and methods of treating vein disorders, malformations, and diseases, particularly varicose vein disorders, malformations, and diseases.

One embodiment of the invention is directed to a pharmaceutical composition comprising a therapeutically effective amount of an amphiphilic block copolymer formulated for administration by injection into a vein. Preferably, the composition is in foam form. The amphiphilic block copolymer is preferably PLURONIC F108®, PLURONIC P105®, PLURONIC F127®, PLURONIC F98®, PLURONIC F87®, PLURONIC F68®, PLURONIC L44®, or combinations thereof. Also preferably, the composition contains a pharmaceutically acceptable carrier.

Particularly preferably, the composition further comprises a co-solvent. The co-solvent is preferably propylene glycol, glycerol, glycerol triacetate, glycerin, polypropylene glycol, polyethylene glycol, tetraglycol, polysorbate-80, other glycols, ethanol, ethyl lactate, absolute ethanol, polidocanol, a polyhydric alcohol, N-methyl 2-pyridoline, dimethyl sulfoxide, a hydrophilic solvent, or combinations thereof. The co-solvent is preferably either a two-agent mixture, or more preferably, in combination with water.

Preferably, the composition further comprises a surfactant. The surfactant is preferably polidocanol, sodium tetradecyl sulfate, or combinations thereof. Preferably, the composition further comprises a vasoconstrictor. The vasoconstrictor is preferably 3-(4,5-dihydro-1H-imidazol-2-ylmethyl)-2,4-dimethyl-6-tert-butyl-phenol (hereinafter, “oxymetazoline”). It is particularly preferable that the composition comprises both a surfactant and a vasoconstrictor.

Preferably, the composition treats spider veins, spider telangiectasias, reticular veins, reticular varicosities, venulectasias, tributary varicose veins, bulging varicose veins, vein tributaries, varicose saphenous veins, or combinations thereof. Preferably, the composition prevents or ameliorates symptoms associated with varicose vein disease. Particularly preferably the symptoms associated with varicose vein disease are edema, skin changes, ulcers, sequelae of varicose veins, bruising, staining, thrombus formation, trapped blood, blood clots, or combinations thereof. Particularly preferably, the symptoms are prevented or ameliorated without use of compression stockings. Preferably, the composition treats venous malformations, arteriovenous malformations, Klippel-Trenaunay Syndrome, aneurysms, endoleaks after aneurysm repair, cerebral aneurysms, tumors, acute bleeding (from trauma), cancer, or combinations thereof. It is also particularly preferable for the composition to further comprise a chemotherapy agent for treating cancer.

Another embodiment of the present invention is directed to a kit comprising the inventive composition. Preferably the kit comprises the inventive composition in a pre-filled syringe or vial.

The invention also relates to a composition comprising a therapeutically effective amount of an agent which is a liquid at about room temperature or below but which is a gel at about skin or body temperature or above, wherein the composition is formulated for administration by injection into a vein for treatment of Varicose Vein Disorder. Preferably the composition comprises a therapeutically effective amount of a surfactant and a vasoconstrictor formulated for administration by injection into a vein. Preferably, the vasoconstrictor is epinephrine, norepinephrine, levophed, dopamine, oxymetazoline, or combinations thereof. The surfactant is preferably polidocanol, sodium tetradecyl sulfate, or combinations thereof. Preferably, the composition further comprises a poloxamer. Particularly preferably, the composition comprises a poloxamer and a co-solvent.

Another embodiment of the present invention is directed to a method for treating Varicose Vein Disorder comprising administering the composition according to embodiments of the invention to a patient. Preferably the method for treating Varicose Vein Disorder comprising injecting a composition comprising oxymetazoline into a vein.

Other embodiments and advantages of the invention are set forth in part in the description, which follows, and in part, may be obvious from this description, or may be learned from the practice of the invention.

### DESCRIPTION OF THE INVENTION

Conventional compositions and methods for the treatment of varicose veins are largely ineffective and pose a high risk of scarring and bruising, and unsightly and potentially irreversible tissue damage. In addition, even successful treatments often lead to complications such as hyperpigmentation of the tissue areas treated.

It was surprisingly discovered that a new class of agents, compositions, products, and methods of treatment directed to Varicose Vein Disease (which includes spider veins, spider telangiectasias, reticular veins, reticular varicosities, venulectasias, tributary varicose veins, bulging varicose veins, vein tributaries, varicose saphenous veins, and as a consequence, edema, skin changes, ulcers and other sequelae of varicose veins) can also eliminate many or all of the side effects and symptoms associated with current treatments. Preferably, embodiments of the invention prevent bruising and/or staining and/or telangiectatic matting and/or thrombus formation, are relatively painless, create a minimal risk of ulceration, do not cause blood to be trapped or blood clot to be formed, and do not require the use of compression stockings. Preferably, pharmaceutical compositions according to various embodiments of the invention are administered according to sclerotherapy methods known by those skilled in the art. The composition may include a variety of pharmaceutically acceptable carriers such as, but not limited to one or more binders, fillers, lubricants, glidants, disintegrants, pharmaceutically acceptable rate controlling polymers, water, alcohol, saline, sugar, glycerol, cellulose, hydrophilic or hydrophobic polymers, buffers, salts and combinations thereof.

Embodiments of this invention are also directed to the treatment of venous malformations, arteriovenous malformations, Klippel-Trenaunay Syndrome, aneurysms, endoleaks after aneurysm repair, cerebral aneurysms, tumors, acute bleeding (from trauma), and cancer. In certain embodiments, malignant masses are treated or shrunk by killing off their blood supply via intra-arterial injection of compositions of this invention. In all embodiments of the invention, preferred ranges for agents comprised in treatment compositions depend upon the type of agent chosen, skin condition, skin type, or a combination of these factors. Compositions according to embodiments can also be manufactured at higher concentrations for subsequent dilution prior to use.

One embodiment of the invention is directed to a varicose vein treatment in which a composition comprising a vasoconstrictor is administered. The vasoconstrictor in certain embodiments can be, for example, epinephrine, norepinephrine, levophed, or dopamine. Preferably, the vasoconstrictor is longer acting, preferably for at least 8 hours, more preferably for at least 10 hours, and most preferably for at least 12 hours. Preferably, the vasoconstrictor employed in certain embodiments is oxymetazoline. Oxymetazoline is currently available and used routinely in research unrelated to sclerotherapy and in over-the-counter cold remedies.

Another embodiment is directed to a composition containing a sclerosant combined with a vasoconstrictor. Preferably, the vasoconstrictor employed is oxymetazoline. The sclerosant can be, for example, sodium tetradecyl sulfate (STS) or polidocanol. Preferably, the sclerosant present in the composition in an amount of from 0.1%-3.0% by weight based on the total weight of the composition. Also preferred are amounts from 0.2% to 1.0%, from 0.5% to 2.5%, from 2% to 3%, from 0.5% to 1.5%, and from 0.1 to 1%. In another embodiment, the composition is converted into a foam of agent(s) combined with air or other combination of gases for administration.

While the various embodiments of the present invention are not bound or limited by any specific theory, the composition according to certain embodiments preferably causes endothelial cell damage when injected into a vein. Additionally, through vasoconstriction, the composition prevents blood from re-entering the vein, and preferably prevents blood clot production, and more preferably also prevents temporary or permanent skin staining. Preferably embodiments have few if any undesirable side effects.

Another embodiment is directed to a composition containing a vasoconstrictor, a poloxamer or other amphiphilic block copolymer, a sclerosant, or any combinations thereof. Preferably, the vasoconstrictor is oxymetazoline. Preferably, refilling of the veins, bruising, thrombus formation, trapped blood and/or skin staining is prevented by administration of compositions according to embodiments of this invention.

Other embodiments of this invention are directed to compositions comprising a poloxamer or a combination of poloxamers. In certain embodiments, a composition comprising a poloxamer or a poloxamer-like composition injures the endothelium with its surfactant/detergent properties and preferably prevents refilling of the treated vein with blood. Preferably, a poloxamer or other nonionic block copolymer that has surfactant properties and that is liquid at room temperature and a gel at body temperature is chosen. While the various embodiments of the present invention are not bound or limited by any specific theory, refilling is preferably prevented in certain embodiments due to the gel form of the poloxamer or other amphiphilic block copolymer of certain embodiments. Preferably, refilling of the veins, bruising, thrombus formation, telangiectatic matting, trapped blood and/or skin staining is also prevented. In another embodiment, a poloxamer or other amphiphilic block copolymer is combined with a sclerosant. In another embodiment, a poloxamer is combined with a vasoconstrictor. In another embodiment, a poloxamer is combined with a sclerosant and a vasoconstrictor. Preferably, the sclerosant in any of the embodiments is sodium tetradecyl sulfate or polidocanol. Preferably, the vasoconstrictor chosen for any of the embodiments is oxymetazoline.

Other embodiments of this invention are directed to compositions comprising a poloxamer, or a combination of poloxamer with a co-solvent. Because the lengths of the polymer blocks can be customized, the poloxamer family contains more than fifty different polymers that have slightly different properties. In certain embodiments, a composition comprising a poloxamer or a poloxamer-like composition preferably injures the endothelium with its surfactant/detergent properties and preferably prevents refilling of the treated vein with blood. Preferably, a poloxamer or other nonionic block copolymer that has surfactant properties is chosen. Particularly preferred poloxamers have surfactant properties that increase the water solubility of hydrophobic substances or increase the miscibility of two substances with different hydrophobicities. Other particularly preferred poloxamers have the property of changing from a liquid to a gel with an increase in temperature. In addition to this reverse thermal behavior, preferable poloxamers may exhibit phase changes (e.g. liquid to gel or gel to liquid) when mixed with both a co-solvent and with water and then subjected to dilution. According to preferred embodiments of the invention, the poloxamer or other nonionic block copolymer is combined with a co-solvent, with or without the addition of water. While the various embodiments of the present invention are not bound or limited by any specific theory, this ternary mixture preferably becomes diluted with blood, plasma, and water upon injection into the vein. This dilution preferably produces gelation of the poloxamer.

Refilling of the vein with blood is preferably prevented in certain embodiments due to the gel form of the poloxamer or other amphiphilic block copolymer of certain embodiments. Preferably, bruising, thrombus formation, telangiectatic matting, trapped blood and/or skin staining is also prevented. In another embodiment, a poloxamer or other amphiphilic block copolymer is combined with a sclerosant. In another embodiment, a poloxamer or other amphiphilic block copolymer is combined with a co-solvent. Preferably, the co-solvent is polidocanol or another surfactant. While the various embodiments of the present invention are not bound or limited by any specific theory, the co-solvent preferably injures the endothelium. Preferably, the poloxamer is combined with a vasoconstrictor. More preferably, the poloxamer is combined with a sclerosant and a vasoconstrictor. The more preferred sclerosant is sodium tetradecyl sulfate or polidocanol. The more preferred vasoconstrictor is oxymetazoline.

In certain embodiments, the poloxamer chosen is one which preferably has the ability to be a liquid at refrigeration temperatures to room temperatures (for example, 0 to 28 degrees Celsius, preferably 18 to 25 degrees Celsius, more preferably 20 to 24 degrees Celsius), but a gel at skin and body temperature (32 to 40 degrees Celsius and higher, preferably 35 to 38 degrees Celsius). While the various embodiments of the present invention are not bound or limited by any specific theory, the poloxamer's gelling properties preferably prevent common side effects associated with vessel destruction. In one embodiment, a composition comprising a poloxamer is injected at room-temperature, in the form of a liquid, into a varicose vein. Upon entering the body and being brought to body temperature, the composition becomes a gel, preferably immediately. Preferably, the poloxamer is a poloxoamer with good gelling properties such as, but not limited to PLURONIC F108®, PLURONIC P105®, PLURONIC F127®, PLURONIC F98®, PLURONIC F87®, PLURONIC® with gel characteristics, PLURONIC® with surfactant and/or detergent properties, and combinations thereof. More preferably, the poloxamer is combined with a surfactant. In one embodiment, the surfactant is sodium tetradecyl sulfate, which is present in the composition in an amount of from 0.1% to 3% by weight based on the total weight of the composition. In another embodiment, the surfactant is polidocanol, and is present in the composition in an amount of from 0.3% to 5% by weight based on the total weight of the composition.

According to preferred embodiments of the present invention, a poloxamer is preferably combined with a co-solvent, and the resulting composition is liquid at room temperature, but converts to a gel soon after injection into a vein. While the various embodiments of the present invention are not bound or limited by any specific theory, the poloxamer's gelling properties preferably prevent common side effects associated with vessel destruction. In one embodiment, a composition comprising a poloxamer is injected at room-temperature, in the form of a liquid, into a varicose vein. Upon entering the body and being diluted by the blood within the vein, the composition becomes a gel, preferably immediately. Poloxamers with good gelling properties include, but are not limited to PLURONIC F108®, PLURONIC P105®, PLURONIC F127®, PLURONIC F98®, PLURONIC F87®, PLURONIC L44®, and combinations thereof. Also preferred are PLURONICS® with gel characteristics, with surfactant and/or detergent properties, and combinations thereof, and preferably the co-solvent allows gelling of the poloxamer to occur soon after injection. More preferably, the co-solvent is selected from, but not limited to propylene glycol, polypropylene glycol, glycerol, glycerol triacetate, glycerin, polyethylene glycol, tetraglycol, polysorbate-80, other glycols, ethanol, ethyl lactate, absolute ethanol, polidocanol, a polyhydric alcohol, N-methyl 2-pyridoline, dimethyl sulfoxide (DMSO), a hydrophilic solvent, and combinations thereof. The co-solvent is preferably either a two-agent mixture, or more preferably, in combination with water.

In certain embodiments, the poloxamer is one which has detergent/surfactant properties. Preferably, the poloxamer chosen has both gelling properties at skin or body temperature of about 30 to 40 degrees Celsius in addition to detergent/surfactant properties. Preferably, the poloxamer with both gelling and surfactant properties is PLURONIC F87®. While the various embodiments of the present invention are not bound or limited by any specific theory, the chosen poloxamer or combination of poloxamers' surfactant properties preferably destroys the endothelium of a vein, while its gelling properties prevent refilling of the vein with blood. Preferably, the poloxamer, or poloxamer-containing agent produces reliable vessel destruction without bruising, trapped blood, thrombus formation, or skin staining. More preferably, these side effects are reduced or eliminated without the patient's needing to wear compression stockings or bandages. Preferably, the poloxamer with good surfactant properties may be, but is not limited to PLURONIC F87®, PLURONIC F68®, PLURONIC F108®, PLURONIC L44®, PLURONIC® with detergent properties, and combinations thereof. In one embodiment, a poloxamer with surfactant properties is combined with a vasoconstrictor, preferably oxymetazoline.

In other embodiments, two different poloxamers are combined to provide both surfactant/detergent properties and gelling properties. In another embodiment, a sclerosant such as sodium tetradecyl sulfate or polidocanol is added to this poloxamer combination.

In other embodiments, a poloxamer, preferably one with gelling properties, is combined with a sclerosant or another locally effective agent. Preferably, the poloxamer chosen also has surfactant properties. In other embodiments, a poloxamer, preferably with surfactant properties, is combined with a vasoconstrictor. Preferably, the poloxamer chosen also has gelling properties.

A PLURONIC® Grid, for example, a grid that represents the relationship between copolymer structure, physical forma, and surfactant characteristics by plotting molecular weight ranges of the hydrophobe against the percent of hydrophile in the final molecule, such as that provided by the PLURONIC® (commercially available from BASF), aids in selecting agents with gelling properties, surfactant properties, or combinations thereof.

Preferably the composition according to the invention is combined with air or a combination of gases and prepared for administration as a foam. The gases may preferably include, but are not limited to O₂, N₂, CO₂, He, H, halides, and other gases that would be apparent to a skilled artisan on the basis of the present disclosure, as well as combinations thereof. For purposes of the present invention the term “foam” refers to an aerated bubble structure created by forcing air into a solution. Preferably, the composition according to the invention is combined with water or saline. Preferably, the composition according to the invention is prepared in a concentration suitable for injection. Particularly preferably, the inventive composition is delivered with a medium-bore needle, angiocatheter, or other catheter into a greater or lesser saphenous vein, a bulging tributary vein, and/or a perforating vein.

One embodiment of the present invention is directed to a composition comprising a poloxamer. Preferably, the composition comprises a poloxamer and a sclerosant. Preferably, the sclerosant may be selected from, but is not limited to sodium tetradecyl sulfate (STS), polidocanol, and combinations thereof. Preferably, the composition comprises a STS in an amount of from 0.1% to 3% by weight based on the total weight of the composition. Preferably, the composition comprises polidocanol in an amount of from 0.3% to 5% by weight based on the total weight of the composition.

Preferably, the composition according to the present invention comprises a poloxamer and a co-solvent. Preferably the co-solvent is propylene glycol with or without water or saline. Particularly preferably the co-solvent is propylene glycol, and is present in the composition in an amount of from 20 to 80% by weight based on the total weight of the composition with or without water or saline.

It is also preferable to employ polidocanol as the co-solvent. Particularly preferably, the composition comprises from 3% to 70% by weight (preferably 5-50% and more preferable 10-35%) based on the total weight of the composition of polidocanol, from 20 to 70% by weight (preferably 5-50% and more preferable 10-35%) based on the total weight of the composition of a poloxamer, and optionally water.

Preferably, the composition comprises a sclerosant. The sclerosant is preferably selected from but not limited to the group sodium tetradecyl sulfate (STS), polidocanol, and combinations thereof. Preferably the composition comprise STS in an amount of from 0.1 to 3% by weight based on the total weight of the composition, or polidocanol in an amount of from 0.3 to 5% by weight based on the total weight of the composition. Particularly preferably, the composition comprises a sclerosant and a vasoconstrictor. The composition preferably comprises a vasoconstrictor in an amount of from 0.0001 to 0.01% by weight based on the total weight of the composition. Particularly preferably, the vasoconstrictor is oxymetazoline.

Preferably, the composition according to the present invention is prepared by mixing the components. Preferably, the mixing is achieved by subjecting the composition to high shear forces. Particularly preferably, the mixing is achieved by rapidly displacing the composition between two syringes. Preferably, each rapid displacement between the two syringes occurs in less than one minute, more preferably in less than 45 seconds, more preferably in less than 30 seconds, more preferably in less than 15 seconds, more preferably in less than 5 seconds, more preferably in less than 1 second. A series of rapid displacements is preferably employed. Each rapid displacement may be of the same or variable duration. The number of rapid displacements is preferably from 1 to 10, 1 to 100, 1 to 1000. Particularly preferably, the two syringes are connected via a stopcock. It is also particularly preferable that the mixing comprising introducing a gas into the composition to produce a foam. The introduction of the gas to produce a foam is preferably achieved due to the high shear forces achieved by rapidly displacing the composition between two syringes. It is often preferable to deliver the composition to a small-bore needle by use of the VARISOLVE® device (commercially available from BTG International, Inc.).

Preferably, pre-filled syringes containing the composition according to the invention are provided to a practitioner as part of a kit. Particularly preferably, a small-bore needle is affixed to the pre-filled syringe. It is also preferable to keep the composition at refrigerator or freezer temperatures (or in an ice/water bath) until injection. Preferably the composition is kept at a temperature of from minus 50 degrees Celsius to 10 degrees Celsius, more preferably from minus 10 degrees Celsius to 5 degrees Celsius

Particularly preferably, the composition according to the present invention is effective for treating cancer, and further comprises a chemotherapy agent or other agent which damages or inhibits cancer cells. Preferably, a treating physician performs arterial puncture and places a delivery catheter into an artery supplying a malignant tumor. Once the catheter tip is in the desired location, the physician injects the inventive composition into the artery, filling the artery and the arteries supplying and intrinsic to the tumor. While the various embodiments of the present invention are not bound or limited by any specific theory, the inventive composition preferably allows prolonged contact of a high concentration of the chemotherapy agent with the tumor, causing enhanced effectiveness in destroying cancer cells while limiting systemic toxicity. It is particularly preferable that the inventive composition possess gel properties.

The following examples illustrate embodiments of the invention, but should not be viewed as limiting the scope of the invention.

### EXAMPLES

### Example 1

A composition comprising from 0.1 to 3% by weight based on the total weight of the composition of sodium tetradecyl sulfate, and from 0.0001 to 0.01% by weight based on the total weight of the composition of oxymetazoline hydrochloride is prepared. The composition is injected by small-bore needle into a varicose vein after the patient's skin has been disinfected, until the blood residing in the vein becomes replaced by the agent, at which point injection ceases and the needle is withdrawn. Repeated injections are made until the abnormal veins, including spider veins, of the patient are treated. In comparison to current methods of sclerotherapy, less, little or no refilling of the vein with blood or bruising is noticeable following the procedure.

### Example 2

A composition containing from 0.1 to 3% by weight based on the total weight of the composition of sodium tetradecyl sulfate, and from 0.0001 to 0.01% by weight based on the total weight of the composition of oxymetazoline hydrochloride is prepared. The composition is converted to a foam by mixing with air through the use of two syringes connected by a stopcock. One syringe is filled with air, one with the composition, and a foam is produced as the contents of the two syringes are mixed by rapid exchanges through the stopcock. The composition is injected by small-bore needle into a varicose vein after the patient's skin has been disinfected, until the blood residing in the vein becomes replaced by the agent, at which point injection ceases and the needle is withdrawn. Repeated injections are made until the abnormal veins, including spider veins, of the patient are treated. Little or no refilling of the vein with blood or bruising is noticeable following the procedure.

### Example 3

A composition is prepared by combining a first solution comprising from 10 to 40% by weight based on the total weight of the first solution of PLURONIC F108® with a second solution comprising from 0.1 to 3% by weight based on the total weight of the second solution of sodium tetradecyl sulfate. The composition is injected by small-bore needle into a varicose vein as described in Example 1. Little or no refilling of the vein with blood or bruising is noticeable following the procedure.

### Example 4

A composition is prepared by combining a first solution comprising from 10 to 40% by weight based on the total weight of the first solution of PLURONIC F108® with a second solution comprising from 0.1 to 10% by weight based on the total weight of the second solution of polidocanol. The composition is injected by small-bore needle into a varicose vein as described in Example 1. Little or no refilling of the vein with blood or bruising is noticeable following the procedure.

### Example 5

A composition comprising from 20 to 70% by weight based on the total weight of the composition of PLURONIC F108®, from 30 to 80% by weight based on the total weight of the composition of propylene glycol, and from 0 to 30% by weight based on the total weight of the composition of water is prepared. The composition is injected by small-bore needle into a varicose vein as described in Example 1. Little or no refilling of the vein with blood or bruising is noticeable following the procedure.

### Example 6

A composition is prepared by combining a first solution comprising from 10 to 40% by weight based on the total weight of the first solution of PLURONIC F127® with a second solution comprising from 0.1 to 3% by weight based on the total weight of the second solution of sodium tetradecyl sulfate. The composition is injected by small-bore needle into a varicose vein as described in Example 1. Little or no refilling of the vein with blood or bruising is noticeable following the procedure.

### Example 7

A composition is prepared by combining a first solution comprising from 10 to 40% by weight based on the total weight of the first solution of PLURONIC F127® with a second solution comprising from 0.1 to 10% by weight based on the total weight of the second solution of polidocanol. The composition is injected by small-bore needle into a varicose vein as described in Example 1. Little or no refilling of the vein with blood or bruising is noticeable following the procedure.

### Example 8

A composition comprising from 20 to 70% by weight based on the total weight of the composition of PLURONIC F127®, from 30 to 80% by weight based on the total weight of the composition of propylene glycol, and from 0 to 30% by weight based on the total weight of the composition of water is prepared. The composition is injected by small-bore needle into a varicose vein as described in Example 1. Little or no refilling of the vein with blood or bruising is noticeable following the procedure.

### Example 9

A composition is prepared by combining a first solution comprising from 10 to 40% by weight based on the total weight of the first solution of PLURONIC F87® with a second solution comprising from 0.1 to 3% by weight based on the total weight of the second solution of sodium tetradecyl sulfate. The composition is injected by small-bore needle into a varicose vein as described in Example 1. Little or no refilling of the vein with blood or bruising is noticeable following the procedure.

### Example 10

A composition is prepared by combining a first solution comprising from 10 to 40% by weight based on the total weight of the first solution of PLURONIC F87® with a second solution comprising form 0.1 to 10% by weight based on the total weight of the second solution of polidocanol. The composition is injected by small-bore needle into a varicose vein as described in Example 1. Little or no refilling of the vein with blood or bruising is noticeable following the procedure.

### Example 11

A composition comprising from 20 to 70% by weight based on the total weight of the composition of PLURONIC F87®, from 30 to 80% by weight based on the total weight of the composition of propylene glycol, and from 0 to 30% by weight based on the total weight of the composition of water is prepared. The composition is injected by small-bore needle into a varicose vein as described in Example 1. Little or no refilling of the vein with blood or bruising is noticeable following the procedure.

### Example 12

A composition comprising from 20 to 70% by weight based on the total weight of the composition of PLURONIC P105®, from 30 to 80% by weight based on the total weight of the composition of propylene glycol, and from 0 to 30% by weight based on the total weight of the composition of water is prepared. The composition is injected by small-bore needle into a varicose vein as described in Example 1. Little or no refilling of the vein with blood or bruising is noticeable following the procedure.

### Example 13

A composition comprising from 20 to 70% by weight based on the total weight of the composition of PLURONIC F87®, from 30 to 60% by weight based on the total weight of the composition of polidocanol, and from 0 to 30% by weight based on the total weight of the composition of water is prepared. The composition is injected by small-bore needle into a varicose vein as described in Example 1. Little or no refilling of the vein with blood or bruising is noticeable following the procedure.

### Example 14

A composition comprising from 20 to 70% by weight based on the total weight of the composition of PLURONIC P105®, from 30 to 60% by weight based on the total weight of the composition of polidocanol, and from 0 to 30% by weight based on the total weight of the composition of water is prepared. The composition is injected by small-bore needle into a varicose vein as described in Example 1. Little or no refilling of the vein with blood or bruising is noticeable following the procedure.

### Example 15

A composition is prepared by combining a first solution comprising from 10 to 40% by weight based on the total weight of the first solution of PLURONIC F87® with a second solution comprising from 0.1 to 3% by weight based on the total weight of the second solution of polidocanol. The composition is injected by small-bore needle into a varicose vein as described in Example 1. Little or no refilling of the vein with blood or bruising is noticeable following the procedure.

### Example 16

A composition comprising from 10 to 40% by weight based on the total weight of the composition of PLURONIC F87® is injected by small-bore needle into a varicose vein as described in Example 1. Little or no refilling of the vein with blood or bruising is noticeable following the procedure.

### Example 17

A composition comprising from 10 to 40% by weight based on the total weight of the composition of PLURONIC F108® is injected by small-bore needle into a varicose vein as described in Example 1. Little or no refilling of the vein with blood or bruising is noticeable following the procedure.

### Example 18

A composition comprising from 10 to 40% by weight based on the total weight of the composition of PLURONIC F98® is injected by small-bore needle into a varicose vein as described in Example 1. Little or no refilling of the vein with blood or bruising is noticeable following the procedure.

### Example 19

A composition comprising from 10 to 40% by weight based on the total weight of the composition of a mixture of two or more PLURONICS®, at least one of said PLURONICS® having gelling properties, and at least another PLURONIC® having surfactant/detergent properties, is injected by small-bore needle into a varicose vein as described in Example 1. Little or no refilling of the vein with blood or bruising is noticeable following the procedure.

### Example 20

A composition is prepared according to any of the preceding examples. An angiocatheter or flexible delivery catheter is placed into the greater saphenous vein, and the composition is delivered into the vein. Little or no refilling of the vein with blood or bruising is noticeable following the procedure.

### Example 21

Gelation and injectability of certain poloxamers and poloxamer/surfactant combinations. Aqueous solutions of selected poloxamers were prepared by weight percentage and refrigerated. After entering solution at refrigeration, the phase and behavior of the solutions was observed at 0 degrees C. and at body temperature (37 degrees C.). Injectability through a 30 gauge needle attached to a 3 cc syringe was assessed as easy (requiring minimal pressure) or by the number of drops expressed per second with moderate pressure on the syringe plunger.

| | State at 0-4 | Injectability at 0-4 | State at body |
|---|---|---|---|
| | degrees C. | degrees C. | temperature |
| F127 20% + | Liquid | easy | gel |
| Polidocanol 3% | | | |
| F127 25% + | Liquid | easy | gel |
| Polidocanol 3% | | | |
| F127 30% + | Liquid | 1.5 secs/drop | gel |
| Polidocanol 3% | | | |
| F108 20% + | Liquid | easy | gel |
| Polidocanol 3% | | | |
| F108 25% + | Liquid | 0.5 secs/drop | gel |
| Polidocanol 3% | | | |
| F108 30% + | Liquid | 1.3 secs/drop | gel |
| Polidocanol 3% | | | |
| F87 30% | Liquid | easy | viscous liquid |
| F87 35% | Liquid | easy | gel |
| F87 40% | Insoluble | — | — |

### Example 22

Injection of certain poloxamer and poloxamer/surfactant combinations into the rabbit marginal ear vein Certain poloxamer and poloxamer/surfactant combinations were prepared as in Example 1, then filtered through a 22 micron filter and maintained sterile in an ice-water bath. One cc syringes with 30 gauge needles were prepared. Under suitable sedation, white New Zealand rabbits were readied, and injections were made into the marginal ear vein on each side. The amount of agent injected with typical manual pressure on the plunger was recorded, and the veins were observed for blood flow and gelation of the injected agent.

| | Injectability | Blood flow | Gelation |
|---|---|---|---|
| F108 27.5% + | 0.5 cc over 3 secs | ceased | present |
| Polidocanol 3% | | | |
| F127 25% + | 0.5 cc over 3 secs | ceased | present |
| Polidocanol 3% | | | |

Other embodiments and uses of the invention will be apparent to those skilled in the art from consideration of the specification and practice of the invention disclosed herein. All references cited herein, including all publications, U.S. and foreign patents and patent applications including U.S. Provisional Application No. 61/027,226, are specifically and entirely incorporated by reference. The term “comprising” as used throughout this application includes the more limiting terms and phrases “consisting essentially of” and “consisting.” It is intended that the specification and examples be considered exemplary only with the true scope and spirit of the invention indicated by the following claims.

1. A pharmaceutical composition comprising a therapeutically effective amount of an amphiphilic block copolymer, wherein the composition is formulated for administration by injection into a vein.

2. The composition of claim 1, wherein the amphiphilic block copolymer is Pluronic F108, Pluronic P105, Pluronic F127, Pluronic F98, Pluronic F87, Pluronic F68, Pluronic L44, or combinations thereof.

3. The composition of claim 1, further comprising a co-solvent.

4. The composition of 3, wherein the co-solvent is selected from the group consisting of propylene glycol, glycerol, glycerol triacetate, glycerin, polypropylene glycol, polyethylene glycol, tetraglycol, polysorbate-80, other glycols, ethanol, ethyl lactate, absolute ethanol, polidocanol, a polyhydric alcohol, N-methyl 2-pyridoline, dimethyl sulfoxide, a hydrophilic solvent, and combinations thereof.

5. The composition of claim 1, which is in a foam form.

6. The composition of claim 1, further comprising a surfactant.

7. The composition of claim 6, wherein the surfactant is selected from the group consisting of sodium tetradecyl sulfate, polidocanol, and combinations thereof.

8. The composition of claim 1, wherein the therapeutically effective amount is effective to treat a condition selected from the group consisting of spider veins, spider telangiectasias, reticular veins, reticular varicosities, venulectasias, tributary varicose veins, bulging varicose veins, vein tributaries, varicose saphenous veins, and combinations thereof.

9. The composition of claim 1, wherein the therapeutically effective amount is effective to prevent or ameliorate a symptom associated with varicose vein disease.

10. The composition of claim 9, wherein the symptom is selected from the group consisting of edema, skin changes, ulcers, sequelae of varicose veins, bruising, staining, thrombus formation, trapped blood, blood clots, and combinations thereof.

11. The composition of claim 1, further comprising a chemotherapy agent.

12. The composition of claim 1, wherein the therapeutically effective amount is effective to treat or ameliorate a condition selected from the group consisting of venous malformations, arteriovenous malformations, Klippel-Trenaunay Syndrome, aneurysms, endoleaks after aneurysm repair, cerebral aneurysms, tumors, acute bleeding, cancer, and combinations thereof.

13. The composition of claim 1 further comprising a therapeutically effective amount of a vasoconstrictor, and a surfactant.

14. The composition of claim 13, wherein the surfactant is selected from the group consisting of sodium tetradecyl sulfate, polidocanol, and combinations thereof.

15. The composition of claim 13, wherein the vasoconstrictor is selected from the group consisting of epinephrine, norepinephrine, levophed, dopamine, oxymetazoline, and combinations thereof.

16. A kit comprising the composition of claim 1 which is provided in a syringe or vial.

17. A composition comprising a therapeutically effective amount of an agent, wherein the agent is a liquid at temperatures between zero and minus 25° C., and wherein the agent is a gel at a temperature of from 32-37° C., and wherein the composition is formulated for administration by injection into a vein for treatment of Varicose Vein Disorder.

18. A kit comprising the composition of claim 17 which is provided in a syringe or vial.

19. A method for treating a varicose vein disorder, the method comprising administering a pharmaceutical composition to a patient, wherein the composition comprises a therapeutically effective amount of an amphiphilic block copolymer, wherein the composition is formulated for administration by injection into a vein.

20. The method of claim 19, wherein the therapeutically effective amount is effective to prevent or ameliorate a symptom associated with the varicose vein disorder.

21. The method of claim 20, wherein the symptom is selected from the group consisting of edema, skin changes, ulcers, sequelae of varicose veins, bruising, staining, thrombus formation, trapped blood, blood clots, and combinations thereof.

22. The method of claim 20, wherein the symptom is prevented or ameliorated without use of compression stockings.

23. A method for treating a varicose vein disorder comprising injecting a pharmaceutical composition comprising oxymetazoline into a vein.
